(19) United States
(12) Patent Application Publication
(10) Pub. No.: US 2024/0188834 A1
(43) Pub. Date: Jun. 13, 2024

(54) APPARATUS AND METHOD FOR MEASURING BLOOD PRESSURE

(71) Applicant: SAMSUNG ELECTRONICS CO., LTD., Suwon-si (KR)

(72) Inventors: Ui Kun KWON, Suwon-si (KR); Young Soo Kim, Suwon-si (KR); Chang Soon Park, Suwon-si (KR); Hye Rim Lim, Suwon-si (KR)

(73) Assignee: SAMSUNG ELECTRONICS CO., LTD., Suwon-si (KR)

(21) Appl. No.: 18/144,645

(22) Filed: May 8, 2023

(30) Foreign Application Priority Data
Dec. 13, 2022 (KR) 10-2022-0173987

(51) Int. Cl. A61B 5/0205; A61B 5/029; A61B 5/11

(52) U.S. Cl. CPC A61B 5/0205; A61B 5/029; A61B 5/1116; A61B 5/02108

(57) ABSTRACT

Provided is an apparatus for estimating blood pressure, including a pulse wave sensor configured to measure a pulse wave signal of a user, and a processor configured to obtain, based on the pulse wave signal, one or more feature values corresponding to the blood pressure, detect whether a change in a posture of a user from a first posture to a second posture occurs while measuring the pulse wave signal, based on the change in the posture being detected, correct a reference blood pressure based on a blood pressure variation caused by the change in the posture, correct the one or more feature values based on a variation in one or more pulse wave signal feature values caused by the change in the posture, and estimate the blood pressure based on the corrected reference blood pressure and the corrected one or more feature values.

### CROSS-REFERENCE TO RELATED APPLICATION

This application claims priority to Korean Patent Application No. 10-2022-0173987, filed on Dec. 13, 2022, in the Korean Intellectual Property Office, the entire disclosure of which is incorporated by reference herein in its entirety.

### BACKGROUND

### 1. Field

Example embodiments of the present disclosure relate to an apparatus and method for non-invasively estimating blood pressure using a pulse wave signal.

### 2. Description of Related Art

With the aging population, increased medical costs, and a lack of medical personnel for specialized medical services, research is being conducted on information technology (IT)-medical convergence technologies, in which IT technology and medical technology are combined. Particularly, monitoring of a health condition of a human body may not be limited to places such as hospitals, but is expanded by mobile healthcare fields that may monitor a user's health condition anywhere (e.g., at home or office on in transit from one place to another place) and anytime in daily life. Some examples of bio-signals, which indicate the health condition of individuals, may include an electrocardiography (ECG) signal, a photoplethysmogram (PPG) signal, an electromyography (EMG) signal, and the like, and various bio-signal sensors are being developed to measure the bio-signals in daily life. For example, the PPG sensor may estimate blood pressure of a human body by analyzing a pulse waveform which reflects a condition of the cardiovascular system and the like.

PPG bio-signal research has shown that the full PPG signal is composed of a superposition of a propagation wave progressing from the heart toward the body distal ends and reflection waves returning from the body distal ends. In addition, it is known that information used to estimate blood pressure can be obtained by extracting various features related to the propagation wave or the reflection waves.

### SUMMARY

One or more example embodiments provide an apparatus and method for non-invasively estimating blood pressure using a pulse wave signal.

According to an aspect of an example embodiment, there is provided an apparatus for estimating blood pressure, including a pulse wave sensor configured to measure a pulse wave signal of a user, and a processor configured to obtain, based on the pulse wave signal, one or more feature values corresponding to the blood pressure, detect whether a change in a posture of a user from a first posture to a second posture occurs while measuring the pulse wave signal, based on the change in the posture being detected, correct a reference blood pressure based on a blood pressure variation caused by the change in the posture, correct the one or more feature values based on a variation in one or more pulse wave signal feature values caused by the change in the posture, and estimate the blood pressure based on the corrected reference blood pressure and the corrected one or more feature values.

The processor may be further configured to detect the change in the posture based on a change in a waveform of the pulse wave signal.

The processor may be further configured to obtain a time interval between two different waveform components included in the pulse wave signal, and detect the change in the posture based on the time interval.

The apparatus may further include at least one of an acceleration sensor, an angular velocity sensor, a gyro sensor, a geomagnetic sensor, or a barometric pressure sensor, wherein the processor may be further configured to detect the change in the posture based on data measured through at least one of the acceleration sensor, the angular velocity sensor, the gyro sensor, the geomagnetic sensor, or the barometric pressure sensor.

The processor may be further configured to correct the reference blood pressure by adding the blood pressure variation caused by the change in the posture to the reference blood pressure, and correct the one or more feature values by subtracting the variation in the one or more pulse wave signal feature values caused by the change the posture from the one or more feature values.

The blood pressure variation caused by the change in the posture may be a general-purpose blood pressure variation including statistical values of blood pressure variations of each of a plurality of users, and the variation in the one or more pulse wave signal feature values caused by the change in the posture may be a variation in one or more general-purpose pulse wave signal feature values including statistical values of variations in one or more pulse wave signal feature values of each of the plurality of users.

The processor may be further configured to correct the general-purpose blood pressure and the variation in the one or more general-purpose pulse wave signal feature values based on at least one of a height of the user, an age of the user, a weight of the user, a gender of the user, or a vascular elasticity of the user.

The blood pressure variation caused by the change in the posture may be a blood pressure variation for each user including a variation in the blood pressure measured in each of the first posture and the second posture of the user, which is caused by the change in the posture, and the variation in the one or more pulse wave signal feature values caused by the change in the posture may be a variation in one or more pulse wave signal feature values for each user including a variation in one or more pulse wave signal feature values measured in each of the first posture and the second posture of the user, which is caused by the change in the posture.

The change in the blood pressure caused by the change in the posture may be a combination of a general-purpose blood pressure variation and a blood pressure variation corresponding to each user, and the variation in the one or more pulse wave signal feature values may be a combination of a variation in the one or more general-purpose pulse wave signal feature values and a variation in one or more pulse wave signal feature values corresponding to each user.

The reference blood pressure may be measured through a cuff at a time of calibration.

The one or more feature values corresponding to the blood pressure may include one or more cardiac output (CO) feature values corresponding to CO and one or more total peripheral resistance (TPR) feature values corresponding to TPR.

The one or more CO feature values may include a ratio between a maximum amplitude value of a pulse wave signal and an area of a predetermined section of the pulse wave signal, and the one or more TPR feature values may include a ratio between an amplitude value of a first reflection wave component of the pulse wave signal and an amplitude value of a propagation wave component of the pulse wave signal.

The processor may be further configured to estimate the blood pressure by linearly combining the corrected reference blood pressure and the corrected one or more feature values.

According to another aspect of an example embodiment, there is provided a method of estimating blood pressure, the method including measuring a pulse wave signal of a user, obtaining, based on the pulse wave signal, one or more feature values corresponding to the blood pressure, detecting whether a change in a posture of a user from a first posture to a second posture occurs while measuring the pulse wave signal, based on the change in the posture being detected, correcting a reference blood pressure based on a blood pressure variation caused by the change in the posture, correcting the one or more feature values based on a variation in one or more pulse wave signal feature values caused by the change in the posture, and estimating the blood pressure based on the corrected reference blood pressure and the corrected one or more feature values.

The detecting whether the change in the posture of the user from the first posture to the second posture occurs may include detecting the change in the posture based on a change in a waveform of the pulse wave signal.

The detecting whether the change in the posture of the user from the first posture to the second posture occurs may include detecting the change in the posture based on data measured through at least one of an acceleration sensor, an angular velocity sensor, a gyro sensor, a geomagnetic sensor, or a barometric pressure sensor.

The blood pressure variation caused by the change in the posture may be a general-purpose blood pressure variation including statistical values of blood pressure variations of each of a plurality of users, and the variation in the one or more pulse wave signal feature values caused by the change in the posture may be a variation in one or more general-purpose pulse wave signal feature values including statistical values of variations in one or more pulse wave signal feature values of each of the plurality of users.

The blood pressure variation caused by the change in the posture may be a blood pressure variation for each user including a variation in blood pressure measured in each of the first posture and the second posture of the user, which is caused by the change in the posture, and the variation in the one or more pulse wave signal feature values caused by the change in the posture may be a variation in one or more pulse wave signal feature values for each user including a variation in one or more pulse wave signal feature values measured in each of the first posture and the second posture of the user, which is caused by the change in the posture.

The change in blood pressure caused by the change in the posture may be a combination of a general-purpose blood pressure variation and a blood pressure variation for each individual, and the variation in the one or more pulse wave signal feature values may be a combination of a variation in the one or more general-purpose pulse wave signal feature values and a variation in one or more pulse wave signal feature values for each individual.

According to yet another aspect of an example embodiment, there is provided an electronic device including a main body, a pulse wave sensor on one surface of the main body, the pulse wave sensor being configured to measure a pulse wave signal from a user, and a processor configured to obtain, based on the pulse wave signal, one or more feature values corresponding to the blood pressure, detect whether a change in a posture of a user from a first posture to a second posture occurs while measuring the pulse wave signal, based on the change in the posture being detected, correct a reference blood pressure based on a blood pressure variation caused by the change in the posture, correct the one or more feature values based on a variation in one or more pulse wave signal feature values caused by the change in the posture, and estimate the blood pressure based on the corrected reference blood pressure and the corrected one or more feature values.

### BRIEF DESCRIPTION OF THE DRAWINGS

Example embodiments will be more clearly understood from the following detailed description taken in conjunction with the accompanying drawings in which:

FIG. 1 is a block diagram illustrating an apparatus for estimating blood pressure according to an example embodiment;

FIGS. 2A and 2B are diagrams illustrating blood pressure for each body part due to the influence of hydrostatic pressure according to posture;

FIG. 3A is a graph for explaining a photoplethysmogram (PPG) waveform in a first posture, and FIG. 3B is a graph for explaining a PPG waveform in a second posture;

FIGS. 4A and 4B are diagrams illustrating a first posture and a second posture;

FIGS. 5A and 5B are diagrams illustrating changes in blood pressure and changes in one or more pulse wave signal feature values when a user's posture is changed from a first posture to a second posture;

FIG. 6 is a block diagram illustrating an apparatus for estimating blood pressure according to another example embodiment;

FIG. 7 is a flowchart illustrating a method of estimating blood pressure according to an example embodiment;

FIGS. 8A, 8B, 9, 10, and 11 are diagrams illustrating various structures of an electronic device including an apparatus for estimating blood pressure.

### DETAILED DESCRIPTION

Details of example embodiments are included in the following detailed description and drawings. Advantages and features of the disclosure, and a method of achieving the same will be more clearly understood from the following embodiments described in detail with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience.

It will be understood that, although the terms first, second, etc. may be used herein to describe various elements, these elements should not be limited by these terms. These terms may be used to distinguish one element from another. Also, the singular forms are intended to include the plural forms as well, unless the context clearly indicates otherwise. It will be further understood that when an element is referred to as “comprising” or “including” another element, the element is intended not to exclude one or more other elements, but to further include one or more other elements, unless explicitly described to the contrary. In the following description, terms such as “unit” and “module” indicate a unit for processing at least one function or operation and they may be implemented by using hardware, software, or a combination thereof. As used herein, expressions such as “at least one of,” when preceding a list of elements, modify the entire list of elements and do not modify the individual elements of the list. For example, the expression, “at least one of a, b, or c,” should be understood as including only a, only b, only c, both a and b, both a and c, both b and c, or all of a, b, and c.

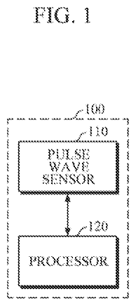

FIG. 1 is a block diagram illustrating an apparatus for estimating blood pressure according to an example embodiment.

Various example embodiments of an apparatus configured to estimate blood pressure described hereinafter may be implemented through an electronic device, such as a smartphone, a tablet personal computer (PC), a desktop PC, a notebook PC, or a wearable device of various forms, such as a wrist watch type, a bracelet type, a wrist band type, a ring type, a glasses type, a hair band type, and the like. In addition, various example embodiments described hereinafter may be applied to estimate various types of cardiovascular information, such as arrhythmias, vascular age, skin elasticity, skin age, arterial stiffness, aortic pressure waveform, stress index, fatigue level, and the like.

Referring to FIG. 1, the apparatus 100 configured to estimate blood pressure includes a pulse wave sensor 110 and a processor 120.

The pulse wave sensor 110 may measure a pulse wave signal including a photoplethysmogram (PPG) signal from a user. In this case, the pulse wave signal may be obtained from a body part of the user which may come into contact with the pulse wave sensor 110, or from a body part of the user where pulse waves may be measured. For example, the body part may include a wrist skin area adjacent to the radial artery and a human body skin area where capillaries or venous blood vessels pass. However, the body part to be measured is not limited thereto, and may be distal body portions, such as fingers and toes, which have a high density of blood vessels.

The pulse wave sensor 110 may include one or more light sources configured to emit light to the body part to be measured and one or more detectors configured to detect reacted light, such as light scattered from or transmitted through an object of interest irradiated by the light source. The light source may include a light emitting diode (LED), a laser diode, a phosphor, and the like. The light source may emit one or more wavelengths of light (e.g., green, red, blue, infrared wavelengths). In addition, the detector may include a photodiode, a photo transistor (PTr), an image sensor (e.g., a complementary metal oxide semiconductor (CMOS) image sensor), or the like, but is not limited thereto.

The processor 120 may be electrically or functionally connected to the pulse wave sensor 110, and may control the pulse wave sensor 110 to acquire a pulse wave signal. The pulse wave sensor 110 may measure a pulse wave signal continuously or at predetermined time intervals (e.g., 1 hour, 2 hours, 4 hours, etc.) under the control of the processor 120. The processor 120 may receive a pulse wave signal from the pulse wave sensor 110 in real time. When receiving a pulse wave signal, the processor 120 may preprocess the pulse wave signal. For example, the processor 120 may perform signal correction, such as filtering (e.g., bandpass filtering at 0.4 to 10 Hz), amplifying a pulse wave signal, converting a pulse wave signal into a digital signal, smoothing, ensemble averaging of continuously measured pulse wave signals, etc. The processor 120 may obtain a representative waveform from the entire waveform of the received pulse wave signal, and analyze the acquired representative waveform to estimate blood pressure. One period of a representative waveform may be extracted using valleys in the entire pulse wave signal waveform and the duration between the valleys. In this case, a plurality of unit waveforms in units of one period may be obtained from the pulse wave signal waveform and any one or a combination of two or more of the plurality of unit waveforms may be obtained as a representative waveform.

The processor 120 may estimate blood pressure by extracting one or more feature values associated with blood pressure from the measured pulse wave signal. In this case, when the change in the user's posture is detected, the estimated blood pressure may be corrected based on a blood pressure variation and a variation in the one or more feature values.

In general, when a user's posture changes at the time of blood pressure estimation through analysis of a pulse wave signal, the hydrostatic pressure of blood vessels in the body changes and the blood pressure changes according to the body part.

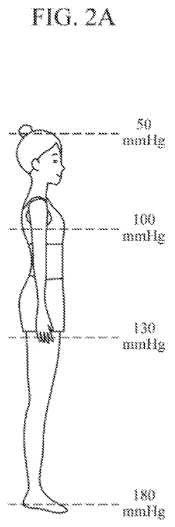

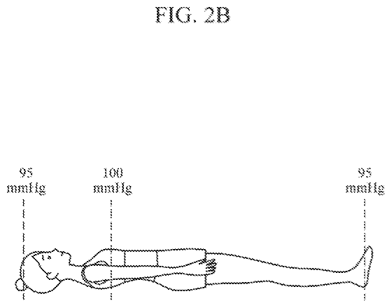

FIGS. 2A and 2B are diagrams illustrating blood pressure for each body part due to the influence of hydrostatic pressure according to posture. For example, FIG. 2A is a diagram illustrating blood pressure for each body part in a standing posture, and FIG. 2B is a diagram illustrating blood pressure for each body part in a lying posture.

For example, in a standing posture of an adult with a blood pressure of 100 mmHg measured at the level of the heart, blood pressure values are estimated as approximately 180 mmHg and approximately 50 mmHg at the feet and head, respectively, due to the hydrostatic pressure. However, in a lying posture, the hydrostatic pressure is similar throughout all blood vessels in the body, and if the blood pressure at the level of the heart is approximately 100 mmHg, which is similar to that in a standing posture, the arterial blood pressure value is similar throughout from the head (blood pressure value: 95 mmHg) to the feet (blood pressure value: 95 mmHg). This indicates that blood pressure changes at each body part due to the hydrostatic pressure as the posture changes.

The pulse wave signal is presented as a superposition of a propagation wave, which propagates along blood vessels when the heart contracts, and reflection waves, which are reflected from the distal end of the body or branching points of the blood vessels, propagate along the blood vessel, and arrive at a point where the pulse wave sensor is located. In this case, when blood pressure at each body part changes due to the change in posture, the pulse wave velocity and reflection coefficient of the pulse wave propagating through the blood vessel also change. As a result, the waveform shape of the pulse wave signal changes due to the change in posture, which may cause an error when the blood pressure is estimated by analyzing the waveform.

For example, since the waveform of the pulse wave signal changes as the user's posture changes, it is necessary to correct the waveform variation of the pulse wave signal along with the blood pressure variation according to the change in posture in order to more accurately estimate the blood pressure.

First, the processor 120 may extract one or more feature values associated with blood pressure from the pulse wave signal measured by the pulse wave sensor. Here, the one or more feature values associated with blood pressure may include one or more cardiac output (CO) feature values associated with CO and one or more total peripheral resistance (TPR) feature values associated with TPR. The feature values associated with blood pressure is not limited to these feature values.

When blood pressure is measured using a general method in a fixed posture, the processor 120 may obtain one or more CO-associated feature values and one or more TPR-associated feature values from the pulse wave signal and estimate the blood pressure based on the obtained one or more CO-associated feature values and the one or more TPR-associated feature values, which may be expressed by Equation 1 and Equation 2 below.

BPₑₛₜ=BP_{cal}+ΔBPₑₛₜ (Equation 1)

BPₑₛₜ=BP_{cal}+function(Δf_{co},Δf_{TPR}) (Equation 2)

Here, BPₑₛₜ is an estimated blood pressure value, BP_{cal} is a reference blood pressure, for example, a blood pressure measured through a cuff at the time of calibration. ΔBPₑₛₜ is a variation in an estimated blood pressure value relative to the time of calibration, and Δf_{co} and Δf_{TPR} are a variation in one or more CO feature values and a variation in one or more TPR feature values relative to the time of calibration, respectively. When there is no change in the user's posture, the user's blood pressure may be estimated through linear combination of the reference blood pressure value at the time of calibration and a function for a blood pressure variation (relative to the time of calibration) estimated from the pulse wave signal, for example, a variation in the one or more CO feature values and a variation in the one or more TRP feature values relative to the time of calibration.

Here, the one or more CO feature values may include a ratio between a maximum amplitude value of a pulse wave signal and an area of a predetermined section of the pulse wave signal, and the one or more TPR feature values may include a ratio between an amplitude value of the first reflection wave component of the pulse wave signal and an amplitude value of the propagation wave component of the pulse wave signal. The one or more CO feature values and the one or more TPR feature values are not limited to the above examples.

Then, the processor 120 may detect whether the user's posture is changed from the first posture to the second posture at the time of measuring the pulse wave signal.

The processor 120 may detect whether the user's posture is changed from the first posture to the second posture on the basis of the waveform change of the pulse wave signal. Here, the first posture may be a standing or sitting posture and the second posture may be a lying posture.

For example, when the pulse wave signal is measured by the pulse wave sensor 110, the processor may calculate a time interval between two different waveform components that constitute the pulse wave signal, compare the time interval with a predetermined threshold, determine that the user's posture is the second posture when the time interval is greater than or equal to the threshold, and determine that the user's posture is the first posture when the time interval is less than the threshold.

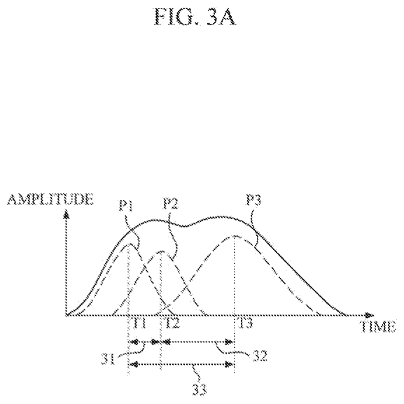

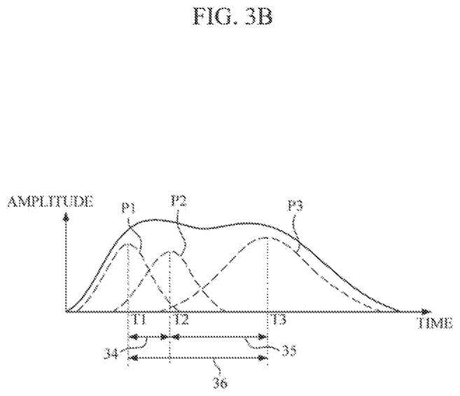

FIG. 3A is a graph for explaining a PPG waveform in a first posture, and FIG. 3B is a graph for explaining a PPG waveform in a second posture.

Referring to FIGS. 3A and 3B, when time intervals between any two different waveform components in the first posture, for example, a time interval 31 between P1 and P2, a time interval 32 between P2 and P3, and a time interval between P1 and P3, are compared to time intervals between any two different waveform components in the second posture, for example, a time interval 34 between P1 and P2, a time interval 35 between P2 and P3, and a time between P1 and P3, respectively, it can be seen that the time interval in the second posture is greater than the time interval in the first posture.

This is because the aortic propagation velocity slows down in the lying posture compared to the standing or sitting posture and hence the time interval between waveform components increases. When a predetermined threshold of the time interval is used, a measurement posture of the user may be determined. For example, when one or more time intervals between any two different waveforms or an average value of the time intervals is greater than or equal to the threshold, it may be determined that the user's posture is a lying posture, and when the one or more time intervals between any two different waveforms or an average value of the time intervals is less than the threshold, it may be determined that the user's posture is a standing or sitting posture. A method of detecting the change in posture based on the waveforms change of the pulse wave signal is not limited to the above examples.

In another example embodiment, the apparatus configured to measure blood pressure may further include at least one of an acceleration sensor, an angular velocity sensor, a gyro sensor, a geomagnetic sensor, or a barometric pressure sensor, and the processor 120 may detect the change in posture based on data measured through at least one of the acceleration sensor, the angular velocity sensor, the gyro sensor, the geomagnetic sensor, or the barometric pressure sensor. For example, the processor 120 may detect the change in the user's posture using the position and direction information measured by the gyro sensor in the apparatus configured to estimate blood pressure.

Then, when the change in posture is detected, the processor 120 may correct the reference blood pressure based on the blood pressure variation caused by the change in posture, and correct the extracted one or more feature values based on the variation in the one or more pulse wave signal feature values caused by the change in posture. Then, the processor 120 may estimate blood pressure by linearly combining the corrected reference blood pressure and the corrected one or more feature values.

Equation 3 below relates to a blood pressure estimation equation based on a pulse wave signal according to another example embodiment.

BPₑₛₜ=BP_{cal}+ΔBPₑₛₜ=BP_{cal}+function(Δf1,Δf2, . . . ,Δfn) (Equation 3)

Here, BPₑₛₜ is an estimated blood pressure value, BP_{cal} is a reference blood pressure measured at the time of calibration, ΔBPₑₛₜ is a variation in the estimated blood pressure value relative to the time of calibration, and Δf1, Δf2, . . . , and Δfn are variations in n feature values extracted from the pulse wave signal waveform relative to the time of calibration.

The calibration may be performed by simultaneously measuring the reference blood pressure and the pulse wave signal at a predetermined point in time, or by measuring alternately the blood pressure and the pulse wave signal one or more times at a predetermined time interval to obtain a statistical value (e.g., average value) of the measured blood pressure as the reference blood pressure and determining a pulse wave signal corresponding to the obtained reference blood pressure.

In general, when blood pressure is measured in a fixed posture, the pulse wave signal measured at the time of calibration and the waveform change of the pulse wave signal at the time of actual measurement may be analyzed to estimate a blood pressure variation according to Equation 3, and a final blood pressure may be estimated by linearly combining the estimated blood pressure variation and the reference blood pressure. In this case, the blood pressure variation may be obtained as a function of the variations Δf1, Δf2, . . . , and Δfn in the n extracted feature values relative to the time of calibration.

As mentioned above, when the user's posture changes, the hemodynamics of the body also changes due to the hydrostatic pressure, and blood pressure may change depending on the body part. In this case, the waveform of the pulse wave signal not only changes due to changes in blood pressure, but also due to factors unrelated to changes in blood pressure, such as hydrostatic pressure and the like. Therefore, a blood pressure estimation method based on Equation 3 may not correspond to the actual blood pressure when the posture changes, and correction is required for the blood pressure.

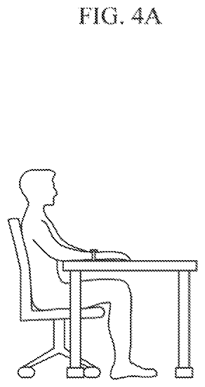

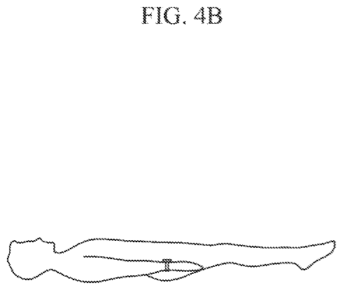

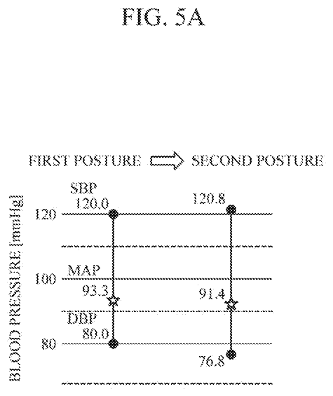

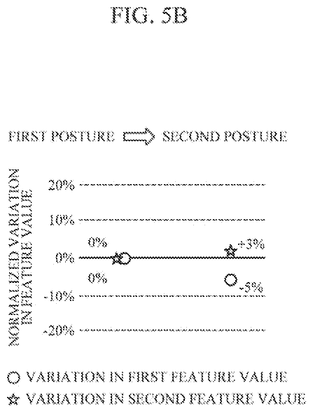

FIGS. 4A and 4B are diagrams illustrating a first posture (sitting posture) and a second posture (lying posture), and FIGS. 5A and 5B are diagrams changes in blood pressure and changes in one or more pulse wave signal feature values when a user's posture is changed from a first posture to a second posture.

Referring to FIG. 5A, when the user's posture is changed from the first posture to the second posture, systolic blood pressure (SBP) changes from 120 mmHg to 120.8 mmHg, diastolic blood pressure (DBP) changes from 80 mmHg to 76.8 mmHg, and mean arterial pressure (MAP) changes from 93.3 mmHg to 91.4 mmHg. In addition, referring to FIG. 5B, when the user's posture is changed from the first posture to the second posture, a variation in the first feature value decreases by approximately 5% and a variation in the second feature value increases by approximately 3%. Thus, when there is a change in posture, the blood pressure and the pulse wave signal may need to be corrected in order to more accurately estimate the blood pressure.

Equation 4 below is obtained by correcting an error due to a change in posture for the pulse wave signal-based blood pressure estimation equation of Equation 3.

BPₑₛₜ=(BP_{cal}+ΔBP_{pc})+function(Δf1−Δf1_{pc}),(Δf2−Δf2_{pc}), . . . ,(Δfn−Δfn_{pc}) (Equation 4)

Here, ΔBP_{pc} is a blood pressure variation caused when the user's posture is changed from the first posture to the second posture, and Δfn_{pc} is a variation in an n^{th} feature value caused when the user's posture is changed from the first posture to the second posture.

According to Equation 4, the processor 120 may correct the reference blood pressure by adding the blood pressure variation caused by the change in posture to the reference blood pressure, and correct the extracted feature values by subtracting the variation in each pulse wave signal feature value caused by the change in posture from each of the extracted feature values.

In this case, the blood pressure variation caused by the change in posture may be a general-purpose blood pressure variation including the statistical value (e.g., an average value) of blood pressure variations of each of a plurality of subjects, and the variation in the one or more feature values of the pulse wave signal caused by the change in posture may be a variation in one or more general-purpose pulse wave signal feature values including the statistical value (e.g., an average value) of the changes in one or more pulse wave signal feature values of each of the subjects.

For example, the general-purpose blood pressure variation and a variation in one or more general-purpose pulse wave signal feature values obtained using a plurality of subjects are stored in the storage of the apparatus configured to estimate blood pressure, and upon receiving a user's request for estimating blood pressure, the processor 120 may estimate the blood pressure through correction according to the change in posture by using the stored general-purpose blood pressure variation and variation in the one or more general-purpose pulse wave signal feature values.

In this case, the processor 120 may correct the general-purpose blood pressure and the variation in one or more general-purpose pulse wave signal feature values based on at least one of the user's height, age, weight, sex, or vascular elasticity and use the corrected general-purpose blood pressure variation and the corrected variation in the one or more general-purpose pulse wave signal feature values.

For example, the plurality of subjects may be classified based on height, age, weight, sex, and vascular elasticity, and accordingly, a regression model may be created based on at least one of height, age, weight, sex, or vascular elasticity. In this case, the processor 120 may input at least one of the user's height, age, weight, sex, or vascular elasticity to the created regression model to obtain the corrected general-purpose blood pressure variation and the corrected variation in the one or more general-purpose pulse wave signal feature values.

In addition, the blood pressure variation caused by the change in posture may be a blood pressure variation for each individual including a variation in blood pressure measured in each of the first posture and second posture of the user, which is caused by the change in posture, and the variation in the one or more pulse wave signal feature values caused by the change in posture may be a variation in the one or more pulse wave signal feature values for each individual including a variation in pulse wave signal feature values measured in each of the first posture and second posture of the user, which is caused by the change in posture.

For example, the blood pressure variation for each individual and the variation in the one or more pulse wave signal feature values for each individual may be measured in advance before blood pressure estimation and stored in the storage of the apparatus configured to estimate blood pressure. Upon receiving the user's request for blood pressure estimation, the processor 120 may estimate the blood pressure through correction according to the change in posture using the stored blood pressure variation for each individual and variation in the one or more pulse wave signal feature values for each individual.

In addition, the change in blood pressure caused by the change in posture may be a combination of the general-purpose blood pressure variation and the blood pressure variation for each individual, and the variation in the one or more pulse wave signal feature values may be a combination of the variation in the one or more general-purpose pulse wave signal feature values and the variation in the one or more pulse wave signal feature values for each individual.

For example, an average value of the general-purpose blood pressure variation and the blood pressure variation for each individual and an average value of the variation in the one or more general-purpose pulse wave signal feature values and the variation in the one or more pulse wave feature values for each individual may be stored in advance in the storage of the apparatus configured to estimate blood pressure, and upon receiving the user's request for estimating blood pressure, the processor 120 may estimate the blood pressure through correction according to the change in posture using the stored average values.

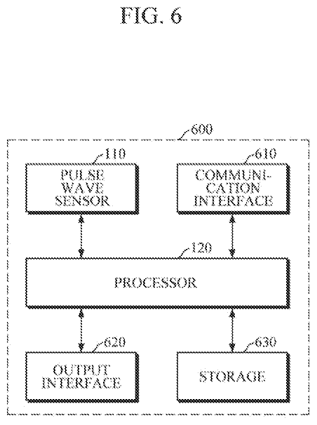

FIG. 6 is a block diagram illustrating an apparatus configured to estimate blood pressure according to another example embodiment.

Referring to FIG. 6, an apparatus 600 configured to estimate blood pressure may include a pulse wave sensor 110, a processor 120, a communication interface 610, an output interface 620, and a storage 630. The pulse wave sensor 110 and the processor 120 are described above in detail, and hence description thereof will not be reiterated hereinafter.

The communication interface 610 may be electrically connected to the processor 120 and may communicate with an external device under the control of the processor 120 to transmit and receive various data, for example, reference blood pressure, various blood pressure estimation equations, and blood pressure estimation results, and the like, using various communication technologies. The external device may include a cuff-type blood pressure measurement device, a smartphone, a tablet PC, a desktop PC, a notebook PC, a wearable device, and the like. However, the external device is not limited to these examples. In this case, the communication technology may include Bluetooth communication, Bluetooth low energy (BLE) communication, near field communication (NFC), wireless local access network (WLAN) communication, ZigBee communication, infrared data association (IrDA) communication, Wi-Fi Direct (WFD) communication, ultra-wideband (UWB) communication, Ant+ communication, Wi-Fi communication, radio frequency identification (RFID) communication, 3G communication, 4G communication, 5G communication, and/or 6G communication.

The output interface 620 may output and provide processing results of the pulse wave sensor 110 and/or the processor 120 to a user. The output interface 620 may provide the information to the user by various visual/non-visual methods using a visual output module including a display, a voice output module such as a speaker, and/or a haptic device providing vibration or tactile sensation.

The storage 630 may store data necessary for the pulse wave sensor 110 and/or the processor 120 and/or processing results of the pulse wave sensor 110 and/or the processor 130. For example, the storage 630 may store blood pressure estimation equations, reference blood pressure, user characteristics (e.g., height, age, weight, sex, and/or vascular elasticity), blood pressure variations caused by change in posture, variations in one or more pulse wave signal feature values caused by change in posture, an estimated blood pressure value, a pulse wave signal generated at the time of blood pressure estimation.

The storage 630 may include at least one type of storage medium of a flash memory type, a hard disk type, a multimedia card micro type, a card type memory (for example, secure digital (SD) or extreme digital (XD) memory), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk, but is not limited thereto.

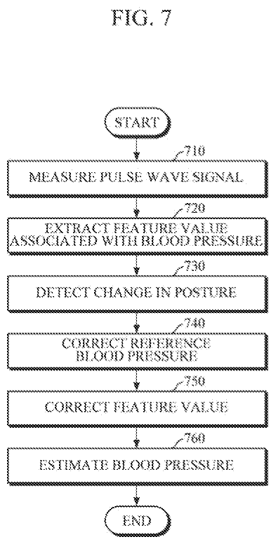

FIG. 7 is a flowchart illustrating a method of estimating blood pressure according to an example embodiment. The method illustrated in FIG. 7 is one example embodiment of a method of estimating blood pressure performed by the apparatuses described above with reference to FIGS. 1 and 6.

First, the apparatus configured to estimate blood pressure may measure a pulse wave signal from a user upon receiving a request for estimating blood pressure in 710.

Then, the apparatus may extract one or more feature values associated with blood pressure from the measured pulse wave signal in 720. Here, the one or more feature values associated with blood pressure may include one or more CO feature values associated with CO, one or more TPR feature values associated with TPR, and the like.

Then, the apparatus may detect whether a user's posture is changed from a first posture to a second posture at the time of measuring the pulse wave signal in 730. In this case, the processor may detect a change in posture based on the change of the waveform of the pulse wave signal. In addition, the apparatus configured to estimate blood pressure may include at least one of an acceleration sensor, an angular velocity sensor, a gyro sensor, a geomagnetic sensor, or a barometric pressure sensor, and may detect a change in posture based on data measured through the sensors.

Then, when the change in posture is detected, the apparatus may correct a reference blood pressure based on a blood pressure variation caused by the change in posture in 740, and correct the extracted one or more feature values based on a variation in one or more pulse wave signal feature values caused by the change in posture in 750. For example, the apparatus may correct the reference blood pressure by adding the blood pressure variation caused by the change in posture to the reference blood pressure, and correct the extracted one or more feature values by subtracting the variation in the one or more pulse wave signal feature values caused by the change in posture from the extracted one or more feature values.

In this case, the blood pressure variation caused by the change in posture may be a general-purpose blood pressure variation including the statistical value of blood pressure variations of each of a plurality of subjects, and the variation in the one or more pulse wave signal feature values caused by the change in posture may be a variation in one or more general-purpose pulse wave signal feature values including the statistical value of variations in one or more pulse wave signal feature values of each of the subjects.

In addition, the blood pressure variation caused by the change in posture may be a blood pressure variation for each individual including a variation in blood pressure measured in each of the first posture and second posture of the user, which is caused by the change in posture, and the variation in the one or more pulse wave signal feature values caused by the change in posture may be a variation in the one or more pulse wave signal feature values for each individual including a variation in one or more pulse wave signal features values measured in each of the first posture and second posture of the user, which is caused by the change in posture.

In another example embodiment, the blood pressure variation caused by the change in posture may be a value obtained by combining the general-purpose blood pressure variation and the blood pressure variation for each individual, and the variation in the one or more pulse wave signal feature values caused by the change in posture may be a value obtained by combining the variation in the one or more general-purpose pulse wave signal feature values and the variation in the one or more pulse wave signal feature values for each individual.

Then, the apparatus may estimate blood pressure based on the corrected reference blood pressure and the corrected one or more feature values in 760. For example, the apparatus may estimate blood pressure by linearly combining the corrected reference blood pressure and the corrected one or more feature values.

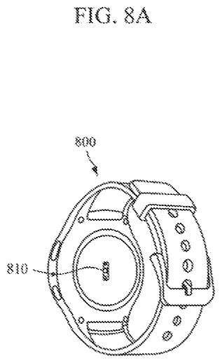

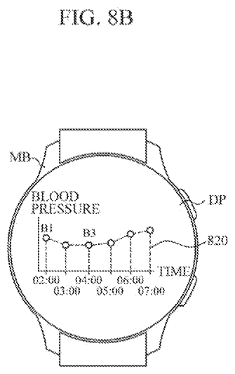

FIGS. 8A to 11 are diagrams illustrating various structures of an electronic device including an apparatus configured to estimate blood pressure.

An electronic device may include a wearable device, for example, a smart watch type, a smart band type, smart eyeglass type, a smart earphone type, a smart ring type, a smart patch, and a smart necklace type, a mobile device, such as a smartphone or a tablet PC, a home appliance, or various types of Internet of Things (IoT) device (e.g., home IoT device, etc.).

The electronic device may include a sensor module, a processor, an input device, a communication module, a camera module, an output device, a storage device, and a power module. The components of the electronic device may be integrally mounted in a specific device, or mounted in two or more devices in a distributed manner. The sensor device may include the pulse wave sensor 110 of the apparatus 100 or 600 estimating blood pressure 100 or 600, and may include additional sensors, such as a gyro sensor, a global positioning system (GPS), an acceleration sensor, an angular velocity sensor, a geomagnetic sensor, a barometric pressure sensor, and the like.

The processor may control the components connected to the processor by executing a program or the like stored in the storage device, and may perform various data processing including blood pressure estimation, or operations. The processor may include a main processor, such as a central processing unit and an application processor, and a co-processor that can be operated independently or together with the main processor, for example, a graphics processing unit, an image signal processor, a sensor hub processor, a communication processor, and the like.

The input device may receive a command and/or data to be used in each component of the electronic device from the user or the like. The input device may include a microphone, a mouse, a keyboard, and/or a digital pen (a stylus pen, etc.).

The communication module may support the establishment of a direct (wired) communication channel and/or wireless communication channel between the electronic device and another electronic device or a server in a network environment or the sensor module and the communication therebetween through the established communication channel. The communication module may be operated independently of the processor 920 and may include one or more communication processors that support direct communication and/or wireless communication. The communication module may include a wireless communication module, such as, a cellular communication module, a short-range wireless communication module, a global navigation satellite system (GNSS) communication module, or the like, and/or a wired communication module, such as a local area network (LAN) communication module, a power line communication module, or the like. Such various types of communication modules may be integrated into a single chip, or may be implemented as a plurality of separate chips. The wireless communication module may verify and authenticate the electronic device in a communication network using subscriber information (e.g., international mobile subscriber identity (IMSI), or the like) stored in a subscriber identity module.

The camera module may capture still images and moving images. The camera module may include a lens assembly including one or more lenses, image sensors, image signal processors, and/or flashes. The lens assembly included in the camera module may collect light emitted from a subject to be imaged.

The output device may output data generated or processed by the electronic device in a visual/non-visual manner. The output device may include a sound output device, a display device, an audio module, and/or a haptic module.

The sound output device may output a sound signal to the outside of the electronic device. The sound output device may include a speaker and/or a receiver. The speaker may be used for general purposes, such as multimedia playback or recording playback, and the receiver may be used to receive incoming calls. The receiver may be combined as part of the speaker or may be implemented as an independent separate device.

The display device may visually provide information to the outside of the electronic device. The display device may include a display, a hologram device, or a projector, and control circuitry to control a corresponding one of the display, hologram device, and projector. The display device may include touch circuitry adapted to detect a touch, or sensor circuitry (e.g., a pressure sensor) adapted to measure the intensity of force incurred by the touch.

The audio module may convert a sound into an electrical signal and vice versa. The audio module may obtain the sound via the input device, or output the sound via the sound output device or a speaker and/or a headphone of an external electronic device directly (e.g., wiredly) or wirelessly coupled with the electronic device.

The haptic module may convert an electrical signal into a mechanical stimulus (e.g., a vibration or motion) or electrical stimulus which may be recognized by a user via his or her tactile sensation or kinesthetic sensation. The haptic module may include, for example, a motor, a piezoelectric element, and/or an electric stimulator.

The storage device may store driving conditions required for driving the sensor module and various data required by other components of the electronic device, for example, software and input data and/or output data for commands related to the software. The storage device may include volatile memory and/or non-volatile memory.

The power module may manage power supplied to the electronic device. The power module may be configured as part of a power management integrated circuit (PMIC). The power module may include a battery and the battery may include a non-rechargeable primary cell, a rechargeable secondary cell, and/or a fuel cell.

Referring to FIG. 8A, the electronic device may be implemented as a smart watch type wearable device 800 and may include a body MB and a wrist strap ST.

The main body MB may be formed to have various shapes. A battery may be embedded in the main body MB and/or the strap ST to supply power to various components of the electronic device. The strap ST may be connected to both ends of the main body MB such that the main body MB may be worn on a user's wrist, and may be flexible be bent around a user's wrist. The strap ST may include a first strap and a second strap separate from the first strap. One ends of the first strap and the second strap may be respectively connected to each end of the main body MB, and the first strap and the second strap may be fastened to each other via fastening means. In this case, the fastening means may be formed as a magnet fastening means, a Velcro fastening means, a pin fastening means, but is not limited thereto. In addition, the strap ST is not limited to the above examples, and may be formed as a single unitary unit such as a non-detachable band.

The main body MB may include a sensor 810, a processor, an output interface, a storage, and a communication interface. However, some of the output interface, storage, and communication interface may be omitted according to the size, shape, and the like of form factor.

The sensor 810 may include a pulse wave sensor configured to measure a pulse wave signal of a user. In addition, the sensor 810 may include at least one of an acceleration sensor, an angular velocity sensor, a gyro sensor, a geomagnetic sensor, or a barometric pressure sensor to detect a change in posture of the user. In this case, the sensor 810 may be disposed on a rear surface of the main body MB to obtain data configured to estimate blood pressure from the wrist by being in contact with an upper portion of the user's wrist when the main body MB is worn on the user's wrist.

The processor mounted in the main body MB may be electrically connected to various components including the sensor 810. For example, the processor may be disposed in the main body MB, may obtain one or more feature values associated with blood pressure from the measured pulse wave signal, detect whether a user's posture is changed from a first posture to a second posture at the time of measuring the pulse wave signal, correct a reference blood pressure based on a blood pressure variation caused by the change in posture when the change in posture is detected, correct the extracted one or more feature values based on a variation in the one or more pulse wave signal feature values caused by the change in posture, and estimate blood pressure based on the corrected reference blood pressure and the corrected one or more feature values.

A display may be provided on a front surface of the main body MB, and various application screens containing the estimated blood pressure value, time information, received message information, and the like may be displayed thereon.

For example, the output interface may display an estimated blood pressure result on the display DP disposed in the main body MB. Referring to FIG. 8B, for example, when blood pressure measurement is automatically performed in units of an hour during nighttime sleeping, the measurement result may be displayed in a visual graph 820. In this case, when the user selects a graphic object B1 on the graph 820, an estimated blood pressure value at a corresponding time (2:00 a.m.) may be displayed on a separate display screen, and when the user selects a graphic object B3, an estimated blood pressure value at a corresponding time (4:00 a.m.) may be displayed on a separate display screen. However, these examples are provided for convenience of explanation, and embodiments are not limited thereto.

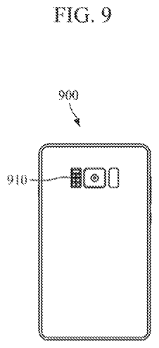

Referring to FIG. 9, the electronic device may be implemented as a mobile device 900 such as a smartphone.

The mobile device 900 may include a housing and a display panel. The housing may form the outer appearance of the mobile device 900. The display panel and cover glass may be sequentially arranged on a first surface of the main body, and the display panel may be exposed to the outside through the cover glass. A sensor 910, a camera module, and/or an infrared sensor may be disposed on a second surface of the housing.

In one example embodiment, a plurality of sensors configured to obtain data from the user may be disposed on the rear surface of a main body of the mobile device 900, and a sensor or the like may be disposed on a fingerprint sensor on the main body, a side power button, or a volume button, or at a separate position on the front and rear surfaces of the mobile device 900 to estimate the blood pressure of the user.

For example, when the user requests estimation of blood pressure by executing an application installed in the mobile device 900, data may be obtained using the sensor 910, the blood pressure may be estimated using a processor in the mobile device. In this case, when there is a change in posture of the user, a reference blood pressure may be corrected based on a blood pressure variation caused by the change in posture, the extracted one or more feature values may be corrected based on a variation in the one or more pulse wave signal feature values caused by the change in posture, and the blood pressure may be estimated based on the corrected reference blood pressure and the corrected one or more feature values.

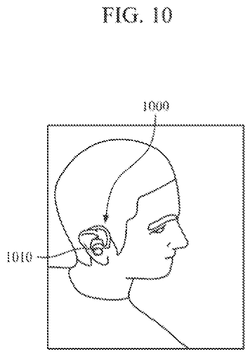

Referring to FIG. 10, the electronic device may be implemented as an ear-wearable device 1000.

The ear-wearable device may include a main body and an ear strap. The user may wear the electronic device by wearing the ear strap on the auricle. The ear strap may be omitted depending on the shape of the ear-wearable device 1000. The main body may be inserted into the external auditory meatus of the user. A sensor 1010 may be mounted in the main body. The ear-wearable device 1000 may acoustically provide an estimation result of blood pressure to the user, or may transmit the estimation result to an external device, for example, a mobile device, a tablet device, a PC, etc. through a communication module provided in the main body.

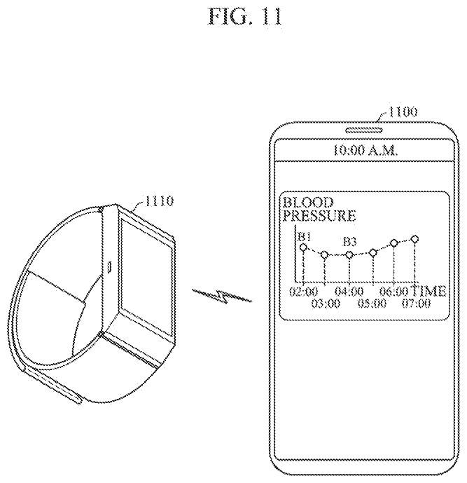

Referring to FIG. 11, the electronic device may be implemented by a combination of a watch-type wearable device and a mobile device such as a smartphone. For example, a processor configured to estimate blood pressure may be mounted in a main body of a mobile device 1100. Upon receiving a request for estimating blood pressure, the processor of the mobile device may communicate with a communication interface mounted in a main body of a wearable device 1110 through a communication interface of the mobile device to control the wearable device to obtain data for estimating blood pressure. In addition, upon receiving the data from the wearable device, the processor may estimate body pressure based on the received data.

The example embodiments can be implemented as computer readable codes in a computer readable record medium. Codes and code segments constituting the computer program can be easily inferred by a skilled computer programmer in the art.

The computer readable record medium includes all types of record media in which computer readable data are stored. Examples of the computer readable record medium include a read-only memory (ROM), a random-access memory (RAM), a CD-ROM, a magnetic tape, a floppy disk, and an optical data storage. Further, the record medium may be implemented in the form of a carrier wave such as Internet transmission. In addition, the computer readable record medium may be distributed to computer systems over a network, in which computer readable codes may be stored and executed in a distributed manner. Functional programs, codes, and code segments for realizing the embodiments can be easily deduced by programmers of ordinary skill in the art, to which the embodiments pertain.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims and their equivalents.

While example embodiments have been described with reference to the figures, it will be understood by those of ordinary skill in the art that various changes in form and details may be made therein without departing from the spirit and scope as defined by the following claims and their equivalents.

What is claimed is:

1. An apparatus for estimating blood pressure, comprising:
a pulse wave sensor configured to measure a pulse wave signal of a user; and
a processor configured to:
obtain, based on the pulse wave signal, one or more feature values corresponding to the blood pressure,
detect whether a change in a posture of a user from a first posture to a second posture occurs,
based on the change in the posture being detected, correct a reference blood pressure based on a blood pressure variation caused by the change in the posture,
correct the one or more feature values based on a variation in one or more pulse wave signal feature values caused by the change in the posture, and
estimate the blood pressure based on the corrected reference blood pressure and the corrected one or more feature values.

2. The apparatus of claim 1, wherein the processor is further configured to detect the change in the posture based on a change in a waveform of the pulse wave signal.

3. The apparatus of claim 2, wherein the processor is further configured to:
obtain a time interval between two different waveform components included in the pulse wave signal, and
detect the change in the posture based on the time interval.

4. The apparatus of claim 1, further comprising:
at least one of an acceleration sensor, an angular velocity sensor, a gyro sensor, a geomagnetic sensor, or a barometric pressure sensor,
wherein the processor is further configured to detect the change in the posture based on data measured through at least one of the acceleration sensor, the angular velocity sensor, the gyro sensor, the geomagnetic sensor, or the barometric pressure sensor.

5. The apparatus of claim 1, wherein the processor is further configured to:
correct the reference blood pressure by adding the blood pressure variation caused by the change in the posture to the reference blood pressure; and
correct the one or more feature values by subtracting the variation in the one or more pulse wave signal feature values caused by the change the posture from the one or more feature values.

6. The apparatus of claim 1, wherein the blood pressure variation caused by the change in the posture is a general-purpose blood pressure variation including statistical values of blood pressure variations of each of a plurality of users, and
wherein the variation in the one or more pulse wave signal feature values caused by the change in the posture is a variation in one or more general-purpose pulse wave signal feature values comprising statistical values of variations in one or more pulse wave signal feature values of each of the plurality of users.

7. The apparatus of claim 6, wherein the processor is further configured to correct the general-purpose blood pressure and the variation in the one or more general-purpose pulse wave signal feature values based on at least one of a height of the user, an age of the user, a weight of the user, a gender of the user, or a vascular elasticity of the user.

8. The apparatus of claim 1, wherein the blood pressure variation caused by the change in the posture is a blood pressure variation for each user including a variation in the blood pressure measured in each of the first posture and the second posture of the user, which is caused by the change in the posture, and
wherein the variation in the one or more pulse wave signal feature values caused by the change in the posture is a variation in one or more pulse wave signal feature values for each user including a variation in one or more pulse wave signal feature values measured in each of the first posture and the second posture of the user, which is caused by the change in the posture.

9. The apparatus of claim 1, wherein the change in the blood pressure caused by the change in the posture is a combination of a general-purpose blood pressure variation and a blood pressure variation corresponding to each user, and
wherein the variation in the one or more pulse wave signal feature values is a combination of a variation in the one or more general-purpose pulse wave signal feature values and a variation in one or more pulse wave signal feature values corresponding to each user.

10. The apparatus of claim 1, wherein the reference blood pressure is measured through a cuff at a time of calibration.

11. The apparatus of claim 1, wherein the one or more feature values corresponding to the blood pressure comprises one or more cardiac output (CO) feature values corresponding to CO and one or more total peripheral resistance (TPR) feature values corresponding to TPR.

12. The apparatus of claim 11, wherein the one or more CO feature values comprise a ratio between a maximum amplitude value of a pulse wave signal and an area of a predetermined section of the pulse wave signal, and
wherein the one or more TPR feature values comprise a ratio between an amplitude value of a first reflection wave component of the pulse wave signal and an amplitude value of a propagation wave component of the pulse wave signal.

13. The apparatus of claim 1, wherein the processor is further configured to estimate the blood pressure by linearly combining the corrected reference blood pressure and the corrected one or more feature values.

14. A method of estimating blood pressure, the method comprising:
measuring a pulse wave signal of a user;
obtaining, based on the pulse wave signal, one or more feature values corresponding to the blood pressure;
detecting whether a change in a posture of a user from a first posture to a second posture occurs;
based on the change in the posture being detected, correcting a reference blood pressure based on a blood pressure variation caused by the change in the posture;
correcting the one or more feature values based on a variation in one or more pulse wave signal feature values caused by the change in the posture; and
estimating the blood pressure based on the corrected reference blood pressure and the corrected one or more feature values.

15. The method of claim 14, wherein the detecting whether the change in the posture of the user from the first posture to the second posture occurs comprises detecting the change in the posture based on a change in a waveform of the pulse wave signal.

16. The method of claim 14, wherein the detecting whether the change in the posture of the user from the first posture to the second posture occurs comprises detecting the change in the posture based on data measured through at least one of an acceleration sensor, an angular velocity sensor, a gyro sensor, a geomagnetic sensor, or a barometric pressure sensor.

17. The method of claim 14, wherein the blood pressure variation caused by the change in the posture is a general-purpose blood pressure variation comprising statistical values of blood pressure variations of each of a plurality of users, and
wherein the variation in the one or more pulse wave signal feature values caused by the change in the posture is a variation in one or more general-purpose pulse wave signal feature values including statistical values of variations in one or more pulse wave signal feature values of each of the plurality of users.

18. The method of claim 14, wherein the blood pressure variation caused by the change in the posture is a blood pressure variation for each user including a variation in blood pressure measured in each of the first posture and the second posture of the user, which is caused by the change in the posture, and
wherein the variation in the one or more pulse wave signal feature values caused by the change in the posture is a variation in one or more pulse wave signal feature values for each user including a variation in one or more pulse wave signal feature values measured in each of the first posture and the second posture of the user, which is caused by the change in the posture.

19. The method of claim 14, wherein the change in blood pressure caused by the change in the posture is a combination of a general-purpose blood pressure variation and a blood pressure variation for each individual, and
wherein the variation in the one or more pulse wave signal feature values is a combination of a variation in the one or more general-purpose pulse wave signal feature values and a variation in one or more pulse wave signal feature values for each individual.

20. An electronic device comprising:
a main body;
a pulse wave sensor on one surface of the main body, the pulse wave sensor being configured to measure a pulse wave signal from a user; and
a processor configured to:
obtain, based on the pulse wave signal, one or more feature values corresponding to the blood pressure,
detect whether a change in a posture of a user from a first posture to a second posture occurs,
based on the change in the posture being detected, correct a reference blood pressure based on a blood pressure variation caused by the change in the posture,
correct the one or more feature values based on a variation in one or more pulse wave signal feature values caused by the change in the posture, and
estimate the blood pressure based on the corrected reference blood pressure and the corrected one or more feature values.
